(19) United States
(12) Patent Application Publication
(10) Pub. No.: US 2025/0204860 A1
(43) Pub. Date: Jun. 26, 2025

(54) ACTIVE CANCELLATION OF NOISE IN A MEDICAL DEVICE

(71) Applicant: KONINKLIJKE PHILIPS N.V., EINDHOVEN (NL)

(72) Inventor: DWAYNE FOLDEN, Bothell, WA (US)

(21) Appl. No.: 18/851,794

(22) PCT Filed: Mar. 28, 2023
(86) PCT No.: PCT/EP2023/058033

§ 371 Date: Sep. 27, 2024

Related U.S. Application Data
(60) Provisional application No. 63/325,768, filed on Mar. 31, 2022.

(51) Int. Cl. A61B 5/00

(52) U.S. Cl. CPC A61B 5/7203; A61B 5/7267

(57) ABSTRACT

Various embodiments an active noise cancellation of the present disclosure encompass measuring and/or monitoring an analog signal acquired for a medical procedure. Unacceptable noise within the analog signal is actively cancelled from the analog via a construction of a noise cancellation signal having a magnitude equal to a magnitude of a sample of the analog signal and further having a phase opposite to the phase of the sample of the analog signal, and injecting the constructed noise cancellation signal into the analog signal. The construction of the noise cancellation signal is conditional of a classification of the analog signal as a standard analog signal or a nonstandard analog signal for a signal measurement application and/or a signal monitoring application in support of the medical procedure.

### FIELD OF THE INVENTION

The present disclosure generally relates to a noise cancellation for an analog signal being monitored and/or measured by a medical device in support of a medical procedure (e.g., diagnostic, therapeutic, surgical, interventional radiology, propaedeutic and anesthesia). The present disclosure specifically relates to an active noise cancellation for such analog signals.

### BACKGROUND OF THE INVENTION

Medical devices as known in the art of the present disclosure for monitoring and/or measuring analog signals in support of a medical procedure include, but are not limited to, Automated External Defibrillators (AEDs), Advanced Life Support (ALS) defibrillators, Basic Life Support (BLS) defibrillators and a variety of patient monitors (e.g., electrocardiogram (ECG) monitors). In practice, the analog signals being monitored and/or measured by such medical devices may experience interference or obstruction do to artefacts or other noise acting on the analog signal. As such, medical devices as known in the art of the present disclosure generally use passive noise filtering of the analog signal in an attempt to remove all of the noise. However, the bandwidth of the passive noise filtering must be designed not to filter out the analog signal, which may result in some, if not all, of the noise passing through the passive noise filtering.

### SUMMARY OF THE INVENTION

The present disclosure describes an active noise cancellation that is applicable to numerous and various medical procedures involving a monitoring and/or measuring of analog signals (e.g., electrocardiography and shock therapy). The active noise cancellation of the present disclosure is premised on classifying an analog signal as a standard analog signal or a nonstandard analog signal for monitoring and/or measuring in support of the medical procedure.

For purposes of describing and claiming the present disclosure, the term “standard analog signal” encompasses a signal that is a standard data input into a signal monitoring application and/or a signal measuring application in support of a medical procedure that results in a reliable monitoring and/or measuring in support of the medical procedure (e.g., noise-free or low noise ECG leads inputted into a ECG monitoring application resulting in a reliable display of an ECG or inputted into an ECG measuring application resulting in an optimal conditional application of a shock therapy).

For purposes of describing and claiming the present disclosure, the term “nonstandard analog signal” encompasses a signal that is a nonstandard data input into a signal monitoring application and/or a signal measuring application of signal in support of a medical procedure that results in an unreliable monitoring and/or measuring in support of the medical procedure (e.g., high noise ECG leads inputted into a ECG monitoring application resulting in a unreliable display of an ECG or inputted into a an ECG measuring application resulting in a sub-optimal conditional application of a shock therapy).

The present disclosure may be embodied as:
(1) An active noise cancellation module for medical devices;
(2) A medical device incorporating an active noise cancellation module; and
(3) An active noise cancellation method for medical devices.

Various active noise cancelling module embodiments of the present disclosure for medical devices encompass a non-transitory machine-readable storage medium encoded with instructions for execution by one or more processors. The non-transitory machine-readable storage medium including the instructions to (1) extract signal information from a sample of an analog signal, (2) classify the analog signal as a standard analog signal or a nonstandard analog signal based on the signal information extracted from the sample of the analog signal, (3) when the sample of the analog signal is classified as a nonstandard analog signal, construct a noise cancellation signal having a magnitude equal to a magnitude of the sample of the analog signal and further having a phase opposite to the phase of the sample of the analog signal, and (4) inject the constructed noise cancellation signal into the analog signal for cancelling the noise within the analog signal.

Various medical device embodiments of the present disclosure in support of a medical procedure encompass a signal application module configured to monitor and/or measure an analog signal, and a noise cancellation module configured to (1) extract signal information from a sample of the analog signal, (2) classify the analog signal as a standard analog signal or a nonstandard analog signal based on the signal information extracted from the sample of the analog signal, (3) when the sample of the analog signal is classified as a nonstandard analog signal, construct a noise cancellation signal having a magnitude equal to a magnitude of the sample of the analog signal and further having a phase opposite to the phase of the sample of the analog signal, and (4) inject the constructed noise cancellation signal into the analog signal for cancelling the noise within the analog signal.

Various active noise cancelling method embodiments of the present disclosure for medical devices in support of a medical procedure involves (1) extracting signal information from a sample of the analog signal, (2) classifying the analog signal as a standard analog signal or a nonstandard analog signal based on the signal information extracted from the sample of the analog signal, (3) when the sample of the analog signal is classified as a nonstandard analog signal, constructing a noise cancellation signal having a magnitude equal to a magnitude of the sample of the analog signal and further having a phase opposite to the phase of the sample of the analog signal, and (4) injecting the constructed noise cancellation signal into the analog signal for cancelling the noise within the analog signal.

The foregoing embodiments and other embodiments of the present disclosure as well as various structures and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

The present disclosure will present in detail the following description of exemplary embodiments with reference to the following figures wherein:

FIG. 1 illustrates a first exemplary embodiment of an active noise cancellation for a medical device in support of a medical procedure in accordance with the present disclosure;

FIG. 2 illustrates a second exemplary embodiment of an active noise cancellation for a medical device in support of a medical procedure in accordance with the present disclosure;

FIG. 3 illustrates an exemplary embodiment of a flowchart representative of an active noise cancellation method a medical device in support of a medical procedure in accordance with the present disclosure;

FIG. 4 illustrates an exemplary embodiment of various components for a medical device in support of a medical procedure in accordance with the present disclosure;

FIG. 5 illustrates a first exemplary embodiment of a medical device in accordance with the present disclosure;

FIG. 6 illustrates a second exemplary embodiment of a medical device in accordance with the present disclosure; and

FIG. 7 illustrates an exemplary embodiment of an active noise cancellation module of FIG. 4 in accordance with the present disclosure.

### DETAILED DESCRIPTION OF THE PREFERRED EMBODIMENTS

An active noise cancellation of the present disclosure for a medical device in support of a medical procedure is applicable to any type of medical procedure involving a monitoring and/or measuring of an analog signal. Examples of such medical procedures include, but are not limited to, diagnostic (e.g., electrocardiography and endoscopy), therapeutic (e.g., shock therapy and respiratory therapy), surgical (e.g., biopsy and endoscopic surgery), interventional radiology, propaedeutic and anesthesia.

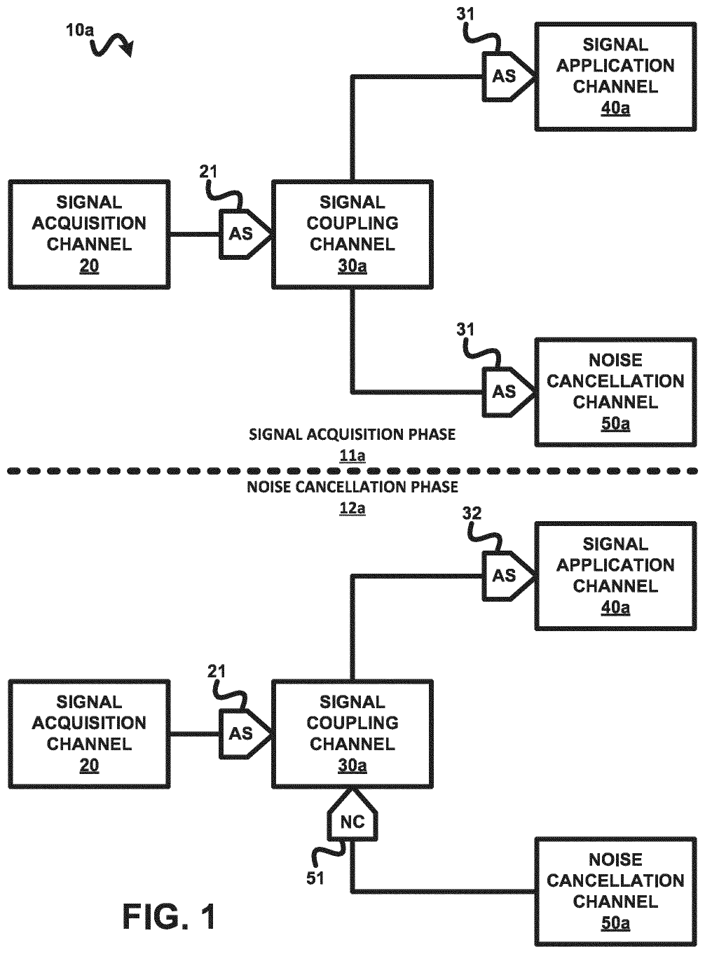

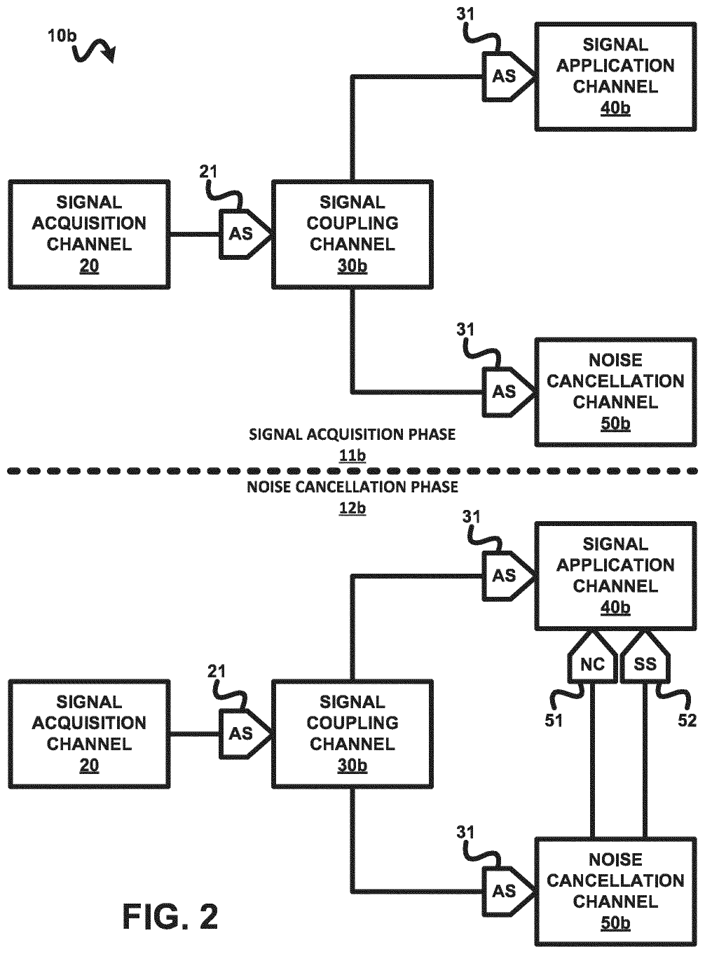

To facilitate an understanding of the present disclosure, the following description of FIGS. 1 and 2 teaches exemplary embodiments of an active noise cancellation for a medical device in support of a medical procedure in accordance with the present disclosure. From the description of FIGS. 1 and 2, those having ordinary skill in the art of the present disclosure will appreciate how to apply the present disclosure to make and use additional embodiments of an active noise cancellation for a medical device in support of a medical procedure in accordance with the present disclosure.

Referring to FIG. 1, a first exemplary embodiment of an active noise cancellation 10a of the present disclosure for a medical device in support of a medical procedure encompasses a signal acquisition channel 20, a signal coupling channel 30a, a signal application channel 40a and a noise cancellation channel 50a.

In an initial implementation of a signal acquisition phase 11a of the active noise cancellation 10a, signal acquisition channel 20 acquires an analog signal 21 relevant to the medical procedure (e.g., an electrocardiogram (ECG) signal) and outputs analog signal 21 to signal coupling channel 30a, which in turn splits the analog signal 21 into analog signal 31 as known in the art of the present disclosure. In practice, analog signal 31 is essentially analog 21 with acceptable coupling loss.

Signal application channel 40a inputs analog signal 31 into a signal monitoring application and/or a signal measuring application in support of the medical procedure (e.g., an ECG monitoring application for displaying an ECG or a ECG measuring application for conditionally applying a shock therapy).

Noise cancellation channel 50a inputs analog signal 31 for purposes of constructing a noise cancellation signal 51 classifies analog signal 31 as a standard analog signal or a nonstandard analog signal for the signal monitoring application and/or the signal measuring application of signal application channel 40a.

Upon classifying analog signal 31 as a nonstandard analog signal, in an initial implementation of a noise cancellation phase 12a of action noise cancellation 10a, noise cancellation channel 50a outputs a constructed noise cancellation signal 51 to signal coupling channel 30a to inject noise cancellation signal 51 into analog signal 21 or analog signal 31 to transform analog signal 31 into a standard analog signal 32 that is inputted into signal application channel 40a for the signal monitoring application and/or the signal measuring application.

Subsequent implementations of signal acquisition phase 11a and noise cancellation phase 12a involve signal application channel 40a processing analog signal 31 whenever noise cancellation channel 50a classifies analog signal 31 as a standard analog signal or processing analog signal 32 whenever noise cancellation channel 50a classifies analog signal 31 as a nonstandard analog signal.

In practice, signal coupling channel 30a may be incorporated into the signal acquisition channel 20.

Referring to FIG. 2, a second exemplary embodiment of an active noise cancellation 10b of the present disclosure for a medical device in support of a medical procedure encompasses signal acquisition channel 20, a signal coupling channel 30b, a signal application channel 40b and a noise cancellation channel 50b.

In an initial implementation of signal acquisition phase 11b of the active noise cancellation 10b, signal acquisition channel 20 acquires an analog signal 21 relevant to the medical procedure (e.g., an electrocardiogram (ECG) signal) and outputs analog signal 21 to signal coupling channel 30b, which in turn splits the analog signal 21 into analog signal 31.

Signal application channel 40b inputs analog signal 31 into a signal monitoring application and/or a signal measuring application in support of the medical procedure (e.g., an ECG monitoring application for displaying an ECG or a ECG measuring application for conditionally applying a shock therapy).

Noise cancellation channel 50b inputs analog signal 31 for purposes of constructing a noise cancellation signal 51 upon classifying analog signal 31 as a standard analog signal or a nonstandard analog signal for the signal monitoring application and/or the signal measuring application of signal application channel 40a.

Upon classifying analog signal 31 as a nonstandard analog signal, in an initial implementation of a noise cancellation phase 12b of action noise cancellation 10b, noise cancellation channel 50b outputs a noise cancellation signal 51 to signal application channel 40b to inject noise cancellation signal 51 into analog signal 31 to transform analog signal 31 into a standard analog signal 32 that is inputted into signal application channel 40a as a standard analog signal for the signal monitoring application and/or the signal measuring application.

To ensure proper cancellation of the noise within analog signal 31, noise cancellation channel 50b also outputs a synchronization signal 52 to facilitate a synchronization of analog signal 31 and noise cancellation signal 51.

Subsequent implementations of signal acquisition phase 11b and noise cancellation phase 12b involve signal application channel 40a only processing analog signal 31 whenever noise cancellation channel 50b classifies analog signal 31 as a standard analog signal or processing analog signal 32 whenever noise cancellation channel 50b classifies analog signal 31 as a nonstandard analog signal.

In practice, signal coupling channel 30b may be incorporated into the signal acquisition channel 20.

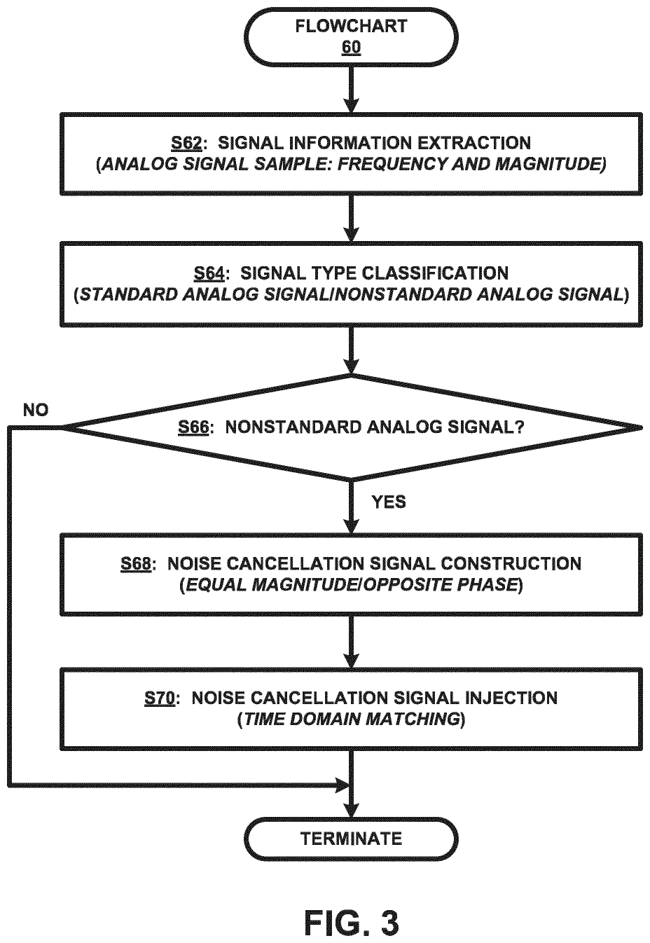

To facilitate a further understanding of the present disclosure, the following description of FIG. 3 teaches an exemplary embodiment of an active noise cancellation method implemented by the noise cancellation channel 50a (FIG. 1) and noise cancellation channel 50b (FIG. 2) in accordance with the present disclosure. From the description of FIG. 3, those having ordinary skill in the art of the present disclosure will appreciate how to apply the present disclosure to make and use additional embodiments of an active noise cancellation method in accordance with the present disclosure.

Referring to FIG. 3, a flowchart 60 is representative of an embodiment of activate noise cancellation method of the present disclosure. The description of flowchart 60 will be provided in the context of the active noise cancellation 10a (FIG. 1) and the active notice cancellation 10b (FIG. 2) as previously described herein.

Referring to FIGS. 1-3, a stage S62 of flowchart 60 encompasses a noise cancellation channel 50 extracting signal information from a sample of analog signal 31. In practice, the signal information must facilitate a classification of analog signal as a standard analog signal or a nonstandard analog signal in accordance with a stage S64 of flowchart 60. In one embodiment of stage S62 as known in the art of the present disclosure, the information extracted from a sample of analog signal 31 includes a time domain of frequency information and of magnitude information of the sample of analog signal 31.

Based on the extracted information of stage S62, stage S64 encompasses a classification of the sample of analog signal 31 as a standard analog signal or a nonstandard analog signal.

Again, for purposes of describing and claiming the present disclosure, the term “standard analog signal” encompasses a signal that is a standard data input into a signal monitoring application and/or a signal measuring application in support of a medical procedure that results in a reliable monitoring and/or measuring in support of the medical procedure (e.g., noise-free or low noise ECG leads inputted into a ECG monitoring application resulting in a reliable display of an ECG or inputted into an ECG measuring application resulting in an optimal conditional application of a shock therapy).

And for purposes of describing and claiming the present disclosure, the term “nonstandard analog signal” encompasses a signal that is a nonstandard data input into a signal monitoring application and/or a signal measuring application of signal in support of a medical procedure that results in an unreliable monitoring and/or measuring in support of the medical procedure (e.g., high noise ECG leads inputted into a ECG monitoring application resulting in a unreliable display of an ECG or inputted into a an ECG measuring application resulting in a sub-optimal conditional application of a shock therapy).

In one embodiment of stage S64 as known in the art of the present disclosure, the time domain of extracted frequency information is converted into a frequency domain of the sample of analog signal 31, which is analyzed to determine if the frequency domain of the sample of analog signal 31 indicates analog signal should be classified as a standard analog signal or as a nonstandard analog signal. In practice, the converted frequency domain of the sample of analog signal 31 may be compared to a profile of a frequency domain suitable for a signal monitoring application and/or a signal measuring application of a signal application channel 40 in support of the medical procedure. Dependent upon the parameters of the comparison, the frequency domain of the sample of analog signal 31, can be classified as a standard analog signal or as a nonstandard analog signal.

In one example for an ECG application, a standard ECG signal has a well understood and structured signal pattern, where the frequency content of a standard ECG signal can be differentiated from nonstandard frequency content.

In practice, machine learning as known in the art of the present disclosure may be trained to distinguish between a standard analog signal and a nonstandard analog signal based on the frequency information and/or magnitude information extracted from the sample of analog signal 31.

Still referring to FIGS. 1-3, if the sample of analog signal 31 is classified as a standard analog signal during stage S64, then a stage S66 of flowchart 60 proceeds to terminate flowchart 60.

Otherwise, if the sample of analog signal 31 is classified as a nonstandard analog signal during stage S64, then stage S66 proceeds to a stage S68 of flowchart 60, which encompasses a construction of a noise cancellation signal 51. In one embodiment of stage S68, noise cancellation signal 51 is constructed to have a magnitude equal to the magnitude of the sample of analog signal 31 and a phase opposite to the phase of the sample of analog signal 31.

A stage 70 of flowchart 60 encompasses an injection of the noise cancellation signal 51 into the analog signal 21 inputted into signal coupling channel 30 or an injection of the noise cancellation signal 51 into the analog signal 31 as inputted into a signal application channel 50.

In one embodiment of stage 570, any repetitiveness of analog signal 31 is determined whereby the noise cancellation signal 51 may be overlapped with the analog signal 21 or analog signal 31 for proper time domain matching. In practice, the time domain matching for an injection the noise cancellation signal 51 into the analog signal 21 acquired from signal acquisition channel 20 involves using the time domain of analog signal 31 as being inputted into a noise cancellation channel 50. Also in practice, the time domain matching for an injection the noise cancellation signal 51 into the analog signal 31 inputted into a signal application channel 50b involves using the time domain of analog signal 31 as being inputted into a noise cancellation channel 50 to generate synchronization signal 52.

Flowchart 60 is terminated upon the injection the noise cancellation signal 51 in accordance with stage S70 whereby the noise cancellation signal 51 is continually injected. Irrespective of whether the first run or a previous run of flowchart 60 classified a sample of analog signal 31 as a standard analog signal or a nonstandard analog signal, flowchart 60 is periodically restarted whereby a new sample of the analog signal 31 will be classified as standard analog signal or a nonstandard analog signal. A noise cancellation signal 51 will be constructed and injected in accordance with stage S70 when the new sample of analog signal 31 is classified as a nonstandard analog signal.

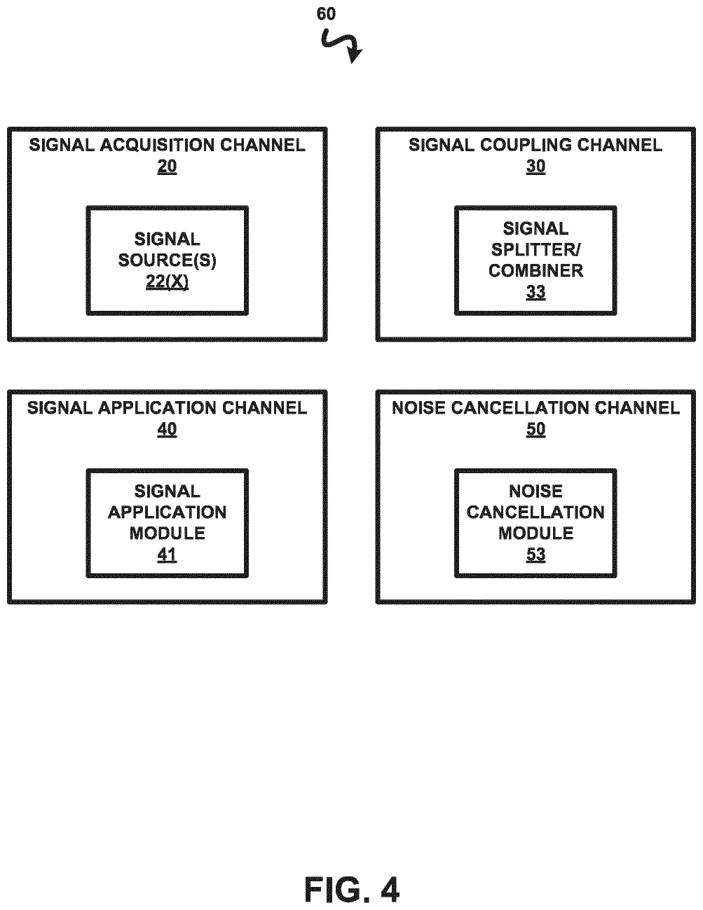

To facilitate a further understanding of the present disclosure, the following description of FIGS. 4-6 teaches an exemplary embodiment of an active noise cancellation medical device in accordance with the present disclosure. From the description of FIGS. 4-6, those having ordinary skill in the art of the present disclosure will appreciate how to apply the present disclosure to make and use additional embodiments of an active noise cancellation medical device in accordance with the present disclosure.

Referring to FIG. 4, medical device components 60 for an active noise cancellation medical device of present disclosure includes an X number of signal source(s) 22(X) implementing signal acquisition channel 20, a signal splitter/combiner 32 implementing signal coupling channel 30, a signal application module 41 implementing signal application channel 40, and noise cancellation module 53 implementing an noise cancellation channel 50.

In practice, signal source(s) 22(X) may be any type of sensor, imager, etc., as known in the art of the present disclosure relevant to the medical procedure including, but not limited to, ECG leads for ECG monitoring on a patient directly. Further, signal source(s) 22(X) may be equipped with ancillary equipment/devices, such as, for example, signal conditioners and passive noise filtering.

In practice, signal splitter/combiner 32 is any type of splitter/combiner as known in the art for splitting and combining analog signals.

In practice, signal application module 41 encompasses an electronic circuit (e.g., electronic components and/or hardware) and/or an executable program (e.g., executable software stored on non-transitory computer readable medium(s) and/or firmware) for executing a specific application associated with the medical procedure. Examples of signal application module 41 include, but are not limited to, ECG measuring module, ECG monitoring modules and conditional shock therapy modules.

In practice, signal application module 53 encompasses an electronic circuit (e.g., electronic components and/or hardware) and/or an executable program (e.g., executable software stored on non-transitory computer readable medium(s) and/or firmware) for executing an active notice cancellation method of the present disclosure, such as, for example, flowchart 60 of FIG. 3.

In practice, an arrangement of signal source(s) 22(X), signal splitter/combiner 32, signal application module 41, and/or noise cancellation module 53 may be arranged in a medical device in support of a medical procedure. In one example, signal splitter/combiner 32 may be incorporated into signal sources (s) 22(X).

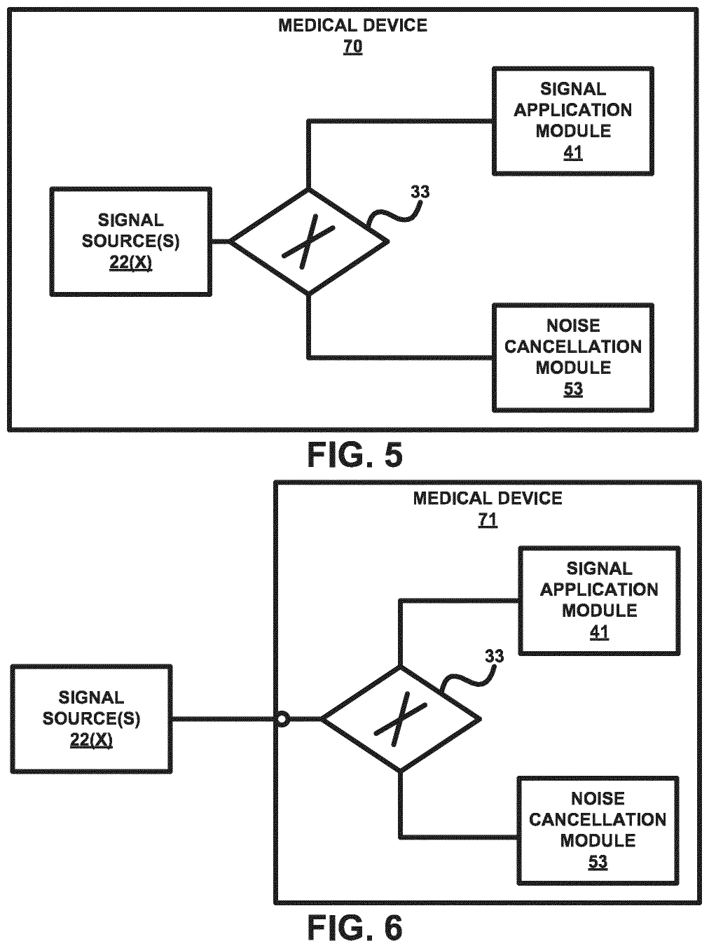

For example, FIG. 5 illustrates a medical device 70 incorporating an arrangement of signal source(s) 22(X), signal splitter/combiner 33, signal application module 41, and noise cancellation module 53 for implementing an active noise cancellation of the present disclosure in support of a medical procedure.

By example, FIG. 6 illustrates a medical device 71 incorporating an arrangement signal splitter/combiner 33, signal application module 41, and noise cancellation module 53 for implementing an active noise cancellation of the present disclosure in support of a medical procedure, whereby signal source(s) 22(X) communicates, wired or wireless, the analog signal to signal splitter/combiner 30.

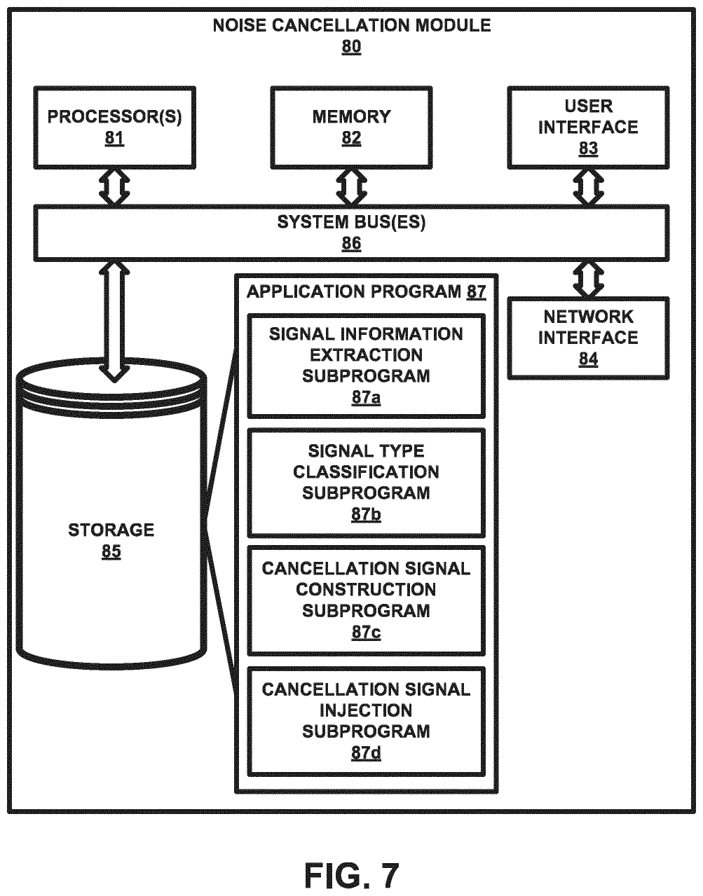

To facilitate a further understanding of the present disclosure, the following description of FIG. 7 teaches an exemplary embodiment of a noise cancellation module in accordance with the present disclosure. From the description of FIG. 7, those having ordinary skill in the art of the present disclosure will appreciate how to apply the present disclosure to make and use additional embodiments of a noise cancellation module in accordance with the present disclosure.

Referring to FIG. 7, an exemplary embodiment 80 of active noise cancellation module 53 that includes one or more processor(s) 81, memory 82, a user interface 83, a network interface 84, and a storage 85 interconnected via one or more system bus(es) 86.

Each processor 81 may be any hardware device, as known in the art of the present disclosure or hereinafter conceived, capable of executing instructions stored in memory 82 or storage or otherwise processing data. In a non-limiting example, the processor(s) 81 may include a microprocessor, field programmable gate array (FPGA), application-specific integrated circuit (ASIC), or other similar devices.

The memory 82 may include various memories, as known in the art of the present disclosure or hereinafter conceived, including, but not limited to, L1, L2, or L3 cache or system memory. In a non-limiting example, the memory 82 may include static random access memory (SRAM), dynamic RAM (DRAM), flash memory, read only memory (ROM), or other similar memory devices.

The user interface 83 may include one or more devices, as known in the art of the present disclosure or hereinafter conceived, for enabling communication with a user such as an administrator. In a non-limiting example, the user interface may include a command line interface or graphical user interface that may be presented to a remote terminal via the network interface 84.

The network interface 84 may include one or more devices, as known in the art of the present disclosure or hereinafter conceived, for enabling communication other components of a medical device. In a non-limiting example, the network interface 84 may include a network interface card (NIC) configured to communicate according to the Ethernet protocol. Additionally, the network interface 84 may implement a TCP/IP stack for communication according to the TCP/IP protocols. Various alternative or additional hardware or configurations for the network interface 84 will be apparent.

The storage 85 may include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage 85 may store instructions for execution by the processor(s) 81 or data upon with the processor(s) 81 may operate. For example, the storage 85 may store a base operating system for controlling various basic operations of the hardware.

The storage 85 also an application program in the form of executable software/firmware for implementing the various functions of active noise cancellation module 53 (FIG. 4) as previously described in the present disclosure. In one exemplary embodiment as shown, storage 85 also stores application program 87 including a signal information extraction subprogram 87a for implementing stage S62 of flowchart 60 (FIG. 3), a signal type identification subprogram 87b for implementing stage S64 and S66 of flowchart 60, a cancellation signal construction subprogram 87c for implementing a stage S68 of flowchart 60, and a cancellation signal injection subprogram 87d for implementing a stage S70 of flowchart 60.

Referring to FIGS. 1-7, those having ordinary skill in the art of the present disclosure will appreciate numerous benefits of the present disclosure including, but not limited to, an active noise cancellation of the present disclosure that overcomes the drawback of passive filtering that allows some noise through the filters (within the passive filter bandwidth) to not remove the wanted analog signal. More particularly, the active noise cancellation of the present disclosure will further reduce the noise passing into a signal application channel and allow for better resolution of the analog information for monitoring and/or measuring purposes. Additionally, the analog signals of the present disclosure may be processed as digital signals at various stages of the active cancellation of the present disclosure.

Further, as one having ordinary skill in the art will appreciate in view of the teachings provided herein, structures, elements, components, etc. described in the present disclosure/specification and/or depicted in the Figures may be implemented in various combinations of hardware and software, and provide functions which may be combined in a single element or multiple elements. For example, the functions of the various structures, elements, components, etc. shown/illustrated/depicted in the Figures can be provided through the use of dedicated hardware as well as hardware capable of executing software in association with appropriate software for added functionality. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of the various and numerous inventions of the present disclosure (which embodiments are intended to be illustrative and not limiting), it is noted that modifications and variations can be made by persons skilled in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device/system or such as may be used/implemented in/with a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. An active noise cancellation medical device, comprising:
a signal application module configured to at least one of monitor and measure an analog signal in support of a medical procedure; and
a noise cancellation module for cancelling noise within the analog signal, wherein the noise cancellation module is configured to:
extract signal information from a sample of the analog signal;
classify the analog signal as a standard analog signal or a nonstandard analog signal based on the signal information extracted from the sample of the analog signal;
when the sample of the analog signal is classified as a nonstandard analog signal, construct a noise cancellation signal having a magnitude equal to a magnitude of the sample of the analog signal and further having a phase opposite to the phase of the sample of the analog signal; and
inject the constructed noise cancellation signal into the analog signal for cancelling the noise within the analog signal.

2. The active noise cancellation medical device of claim 1, wherein the noise cancellation module includes a non-transitory machine-readable storage medium encoded with instructions for execution by at least one processor, the non-transitory machine-readable storage medium for cancelling noise within the analog signal including the instructions to:
extract signal information from a sample of the analog signal;
classify the analog signal as the standard analog signal or the nonstandard analog signal based on the signal information extracted from the sample of the analog signal;
when the sample of the analog signal is classified as a nonstandard analog signal, construct a noise cancellation signal having a magnitude equal to a magnitude of the sample of the analog signal and further having a phase opposite to the phase of the sample of the analog signal; and
inject the constructed noise cancellation signal into the analog signal for cancelling the noise within the analog signal.

3. The active noise cancellation medical device of claim 1, wherein the noise cancellation module is configured to extract at least one of frequency information and magnitude information from the sample of the analog signal.

4. The active noise cancellation medical device of claim 1, wherein the noise cancellation module is configured to implement machine learning trained on the signal information to classify the analog signal as the standard analog signal or the nonstandard analog signal based on the signal information extracted from the sample of the analog signal.

5. The active noise cancellation medical device of claim 1, wherein the noise cancellation module is configured to implement time domain matching to synchronize an injection of the constructed noise cancellation signal into the analog signal for cancelling the noise within the analog signal.

6. The active noise cancellation medical device of claim 1, further comprising:
a signal splitter/combiner configured to communicate the analog signal to the signal application module and the noise cancellation module.

7. The active noise cancellation medical device of claim 6, further comprising:
at least one signal source configured to communicate the analog signal to the signal splitter/combiner.

8. A noise cancellation module, comprising:
a non-transitory machine-readable storage medium encoded with instructions for execution by at least one processor, the non-transitory machine-readable storage medium for cancelling noise within an analog signal including the instructions to:
extract signal information from a sample of the analog signal;
classify the analog signal as a standard analog signal or a nonstandard analog signal based on the signal information extracted from the sample of the analog signal;
when the sample of the analog signal is classified as a nonstandard analog signal, construct a noise cancellation signal having a magnitude equal to a magnitude of the sample of the analog signal and further having a phase opposite to the phase of the sample of the analog signal; and
inject the constructed noise cancellation signal into the analog signal to transform the analog signal into the standard analog signal for cancelling the noise within the analog signal.

9. The noise cancellation module of claim 8, wherein the signal information is at least one of frequency information and magnitude information extracted from the sample of the analog signal.

10. The noise cancellation module of claim 8, wherein a machine learning trained on the signal information classifies the analog signal as the standard analog signal or the nonstandard analog signal based on the signal information extracted from the sample of the analog signal.

11. The noise cancellation module of claim 8, wherein a time domain matching serves as a basis for a synchronization an injection of the constructed noise cancellation signal into the analog signal for cancelling the noise within the analog signal.

12. An active noise cancellation method for cancelling noise within an analog signal in support of a medical procedure, the active noise cancellation method comprising:
extracting signal information from a sample of the analog signal;
classifying the analog signal as a standard analog signal or a nonstandard analog signal based on the signal information extracted from the sample of the analog signal;
when the sample of the analog signal is classified as a nonstandard analog signal, constructing a noise cancellation signal having a magnitude equal to a magnitude of the sample of the analog signal and further having a phase opposite to the phase of the sample of the analog signal; and
injecting the constructed noise cancellation signal into the analog signal for cancelling the noise within the analog signal.

13. The active noise cancellation method of claim 12, wherein the signal information is at least one of frequency information and magnitude information extracted from the sample of the analog signal.

14. The active noise cancellation method of 12, wherein a machine learning trained on the signal information classifies the analog signal as the standard analog signal or the nonstandard analog signal based on the signal information extracted from the sample of the analog signal.

15. The active noise cancellation method of 12, wherein a time domain matching serves as a basis for a synchronization of an injection of the constructed noise cancellation signal into the analog signal for cancelling the noise within the analog signal.
